SEQUENCE LISTING (2 sequences)

SEQ ID NO: 1 - DNA, 60 bp, Artificial Sequence
  505F/806R forward primer
aatgatacggcgaccaccgagatctacactatggtaattgtgtgccagcmgccgcggtaa

SEQ ID NO: 2 - DNA, 56 bp, Artificial Sequence
  505F/806R Reverse primer
caagcagaagacggcatacgagatagtcagtcagccggactachvgggtwtctaat

(19) United States
(12) Patent Application Publication
(10) Pub. No.: US 2019/0360022 A1
(43) Pub. Date: Nov. 28, 2019

(54) COMPOSITION CONTAINING INTRAVAGINAL MICROORGANISMS

(71) Applicant: Seoul National University R&DB Foundation, Seoul (KR)

(72) Inventors: GwangPyo KO, Seoul (KR); Jiyeon SI, Ansan-si (KR); Hyun Ju YOU, Incheon (KR)

(21) Appl. No.: 16/330,946

(22) PCT Filed: Sep. 18, 2017
(86) PCT No.: PCT/KR2017/010202

§ 371 Date: Mar. 6, 2019

(30) Foreign Application Priority Data
Sep. 27, 2016 (KR) 10-2016-0124109

(51) Int. Cl. C12Q 1/689; G01N 33/50

(52) U.S. Cl. CPC C12Q 1/689; G01N 33/502; C12Q 2600/112; G01N 2800/50; G01N 2800/044; G01N 2800/36; C12Q 2600/118

(57) ABSTRACT

The present invention relates to a biomarker of predicting or diagnosing female genital disease or obesity. More specifically, the present invention relates to a composition for predicting or diagnosing risk of female genital disease or obesity, including detecting Prevotella spp., Sneathia spp. Megasphaera spp., Gardnerella spp., and Lactobacillus spp.

### TECHNICAL FIELD

The present invention relates to the selection and use of biomarkers for predicting or diagnosing the risk of female genital diseases or obesity by utilizing vaginal microorganisms.

### RELATED ART

In all over the world, three-quarters of the female population have being experiencing an infection of inflammatory female disease, including bacterial vaginitis, of which 50% are reported to experience recurrence. In addition, more than 60% of female vaginitis is asymptomatic infection, and the susceptibility to vaginitis is greatly increased due to antibiotics, stress, and hormonal changes after menopause.

The causes of infection-induced vaginitis are divided into three major causes: bacterial vaginosis (BV), candida vaginitis (CV), and Trichomonas vaginalis. Bacterial vaginitis is the most common vaginitis in recent years, and vaginal secretions from patients with bacterial vaginosis have been reported to include Atopobium vaginae, Megasphaera spp., Gardnerella vaginalis, Eggerthella genus, Clostridium-like genus, Prevotella bivia, Peptostreptococcus micros, and the like have been found, Gardnerella vaginalis is a major cause of bacterial vaginitis.

Female vaginal microbiota is affected by various factors such as race, age, pregnancy/birth/feeding, pathogen infection, lifestyle, immune and metabolic abnormalities, and the like. Especially, in case of typical female diseases including vaginitis, significant changes are observed in the colony structure of bacterial microbiota in the normal vagina, and recovery of the destructed bacterial balance is essential for the prevention and treatment of female diseases. The key indicators such as the type of Lactobacillus group (subdividing adult female normal microflora into five groups) and acidity of vaginal environment in healthy vagina are also different according to the genetic characteristics and race of an individual.

Through the analysis of microorganisms using the next generation sequencing method can identify all microorganism species including non-culturing microorganisms. Such method is to identify microbial species using 16s ribosomal RNA gene (16S rRNA) which are conserved in all bacteria, and the hypervariable region, and to identify the relationship between microbial genome diversity and the disease. These microbial assays have liberated the relationship between existing bacteria and diseases from the conventional limit of cultivation possibility, and have made a great improvement in that they have been reevaluated from the whole point of view. In particular, they have proved the epidemic relationship between intestinal microorganisms and obesity.

In the past, studies on the relationship or mechanism between human microbiome and health have been conducted mainly on intestinal microorganisms. It has been speculated that the female vaginal microbiota are directly related to female genital health and have a different microbiota depending on the race. To date, the studies of the disease and the genetic effects on human microbiome have been limited to intestinal microbiome, but have not been reported the female vaginal microbiome as yet.

### DISCLOSURE

### Technical Problem

The present invention relates to a composition for diagnosing, preventing or treating female genital diseases, including an agent of a detectable agent of at least a vaginal microorganism selected from the group consisting of Prevotella spp., Sneathia spp. Megasphaera spp., Gardnerella spp., and Lactobacillus spp, and a kit for predicting or diagnosing risk of female genital diseases by using at least a vaginal microorganism.

The present invention relates to a composition for diagnosing, preventing or treating obesity, including an agent of a detectable agent of at least a vaginal microorganism selected from the group consisting of Prevotella spp., Sneathia spp. Megasphaera spp., Gardnerella spp., and Lactobacillus spp, and a kit for predicting or diagnosing risk of obesity by using at least a vaginal microorganism.

### Technical Solution

In order to achieve the object, the inventors of the present invention have conducted extensive studies to determine the microbiota present in the female cervix by using a large-scale sequencing analysis and to examine the relationship with female genital diseases or obesity, so as to develop a composition or a kit for prevent/treating female genital diseases or obesity.

An embodiment of the present invention is a composition for predicting or diagnosing the risk of female genital diseases, a kit comprising the same, or a method of detecting microorganisms in vagina, in order to provide information on prediction or diagnosis of risk of female genital diseases.

An embodiment of the present invention is a composition for predicting or diagnosing the risk of obesity, a kit comprising the same, or a method of detecting microorganisms in vagina, in order to provide information on prediction or diagnosis of risk of obesity.

According to the composition of the present invention, it can be applied as a biomarker that can be used for prediction, prevention, diagnosis, or treatment of female genital diseases or obesity by using vaginal microbiome in female vagina.

Hereinafter, the present invention will be described in more detail.

An embodiment of preset invention relates to a composition for predicting or diagnosing risk of female genital diseases, comprising a detectable agent of at least a vaginal microorganism selected from the group consisting of Prevotella spp., Sneathia spp. Megasphaera spp., Gardnerella spp., and Lactobacillus spp.

The female genital diseases to be applied by the composition may be at least one selected from the group consisting of vaginitis, menopause, cervical inflammation and cervical cancer, more preferably menopausal and/or vaginitis.

An embodiment of preset invention relates to a composition for predicting or diagnosing risk of obesity, comprising a detectable agent of at least a vaginal microorganism selected from the group consisting of Prevotella spp., Sneathia spp. Megasphaera spp., Gardnerella spp., and Lactobacillus spp.

Obesity is a phenomenon in which excessive accumulate state of fat in the body and the leftover calories are converted to fat and accumulate in the subcutaneous tissues or abdominal intermembrane in the human body. The composition of the present invention relates to prediction or diagnosis of the obesity risk by using not intestinal microorganisms but vaginal microorganism distributed in the female genitalia. Particularly, it is possible to predict or diagnose the risk of obesity at higher sensitivity, by using vaginal microorganism.

The composition of the present invention is characterized in that the target microorganisms to be detected of the composition is microorganisms originating from the female vagina, wherein the microorganisms belonging to Prevotella spp., Sneathia spp. Megasphaera spp., Gardnerella spp., and Lactobacillus spp. Accordingly, the composition is characterized in that it is applied to a sample collected from female vagina.

Preferably, Lactobacillus is a vaginal dominant species that secretes lactic acid or a sterile liquid to protect the female genital organ from harmful bacteria. It has been reported that Prevotella spp. is resident flora in oral and genital organ and is directly related to the vaginal inflammatory response according to recent studies. Sneathia spp. was initially classified with Leptotrichia but was separately isolated as a strain of Sneathia spp. Sneathia spp. has been reported to be associated with genital diseases such as abortion, prematurity, amnionitis, and vaginitis. Gardnerella spp. has only one species of G. vaginalis, which was initially classified as Haemophilus vaginalis, later classified as Corynebacterium vaginalis, and finally classified as Gardnerella spp. Gardnerella spp. has both properties of Gram-negative or Gram-positive, and has been detected not only in female genitalia but also in blood, urine, and pharynx.

Particularly, Prevotella spp., Sneathia spp. and Gardnerella spp. have negative correlation with Lactobacillus, which is the dominant species in vaginal flora, in female genital diseases or obesity.

Herein, the term “diagnostic marker or marker for diagnosing” refers to a reference substance that distinguish a female genital disease or an obese state from normal state, and includes various organic biomolecules that exhibit an increase or decrease in activity at a sample of patient with a female genital disease or obesity, being compared to the normal sample. For the purpose of the present invention, the diagnostic composition of the present invention refers to Prevotella spp., which is expressed at an especially high level in both samples of patient with female genital diseases and patients with obesity, and a microorganism or microbial group of Sneathia spp. Megasphaera spp., and/or Gardnerella spp which have positive correlation with Prevotella spp.

In the past, studies on the relationship or mechanism between human microbiome and health have been conducted mainly on intestinal microorganisms. The female vaginal microbiota are directly related to female genital health and have a different microbiota depending on the race, so as to be presumed to have a large genetic influence. To date, the studies of the disease and the genetic effects on human microbiome have been limited to intestinal microbiome, but have not been reported the female vaginal microbiome as yet. However, the present invention is based on the finding that microorganisms derived from vagina can be used as a sample and thus it is possible to predict or diagnose female genital diseases and obesity.

Preferably, the detectable agent refers to a substance that can be used for detecting the presence of Prevotella spp., Sneathia spp. Megasphaera spp., Gardnerella spp., a diagnostic biomarker of female genital diseases or obesity in a sample. For example, the detectable agent may be at least one selected from the group consisting of a primer, a probe, antisense oligonucleotide, aptamer and antibody which can specifically detect organic biomolecules such as a protein, a nucleic acid, a lipid, a glycolipid, a glycoprotein or a saccharide (monosaccharide, a disaccharide, a disaccharide, or a polysaccharide), and the like.

In the present invention, the detectable agent of microorganism may be an antibody, and the microorganism may be detected using an immunological method based on an antigen-antibody reaction. The analysis methods include Western blotting, enzyme linked immunosorbent as (ELISA), radioimmunoassay (RIA), radioimmunodiffusion, Ouchterlony immunodiffusion, rocket immunoassay Immunoprecipitation assays, complement fixation assays, fluorescence activated cell sorters (FACS), protein chips, and the like, but not limited thereto.

Preferably, the agent capable of detecting the microorganism is a microorganism-specific primer. The term, “primer” refers to a primer capable of forming a base pair complementary to a template strand, and to a nucleotide sequence of 7 to 50 bases functioning as a starting point for replicating the template. The primers are usually synthesized, but may be used as naturally occurring nucleotide sequence. The sequence of the primer does not necessarily have to be exactly the same as the sequence of the template, but is sufficiently complementary so long as it can hybridize with the template. Additional features that do not alter the basic properties of the primer can be incorporated to the primers. Examples of the additional features include, but are not limited to, methylation, capping, substitution of one or more nucleotides with its derivatives, and the nucleotide modification.

Preferably, the primer used in the present invention may be a primer capable of amplifying a 16S rRNA of a strain belonging to Prevotella spp., Sneathia spp. Megasphaera spp., Gardnerella spp. For example, the primer may be the primer shown in Table 1.

TABLE 1

| | SEQ | |
|---|---|---|
| | ID | |
| Name | NO | Nucleotide sequence (5′-->3′) |
| 505F/806R | 1 | AATGATACGGCGACCACCGAGATCTACACT |
| Forward primer | | ATGGTAATTGTGTGCCAGCMGCCGCGGTAA |
| 505F/806R | 2 | CAAGCAGAAGACGGCATACGAGATAGTCAG |
| Reverse primer | | TCAGCCGGACTACHVGGGTWTCTAAT |

The 16s rRNA is a 30S subunit of the prokaryotic ribosome. Most of the 16s rRNA is conserved in the rRNA, while some regions exhibit high sequence diversity. In particular, there is little diversity among same species, but large diversity among different species, so that the sequence of 16S rRNA can be compared to identify prokaryotes effectively.

The risk prediction is to determine whether the subject is likely to develop female genital diseases or obesity and to identify the risk of female genital diseases or obesity. They can be used clinically to make treatment decisions by delaying the onset of the disease or preventing it from developing, or by selecting the most appropriate treatment method. In addition, the term “diagnosis” means the confirmation of the presence or characteristic of a pathological condition. For the purpose of the present invention, the diagnosis may mean the confirmation on whether or not female genital disease or obesity has occurred.

A preferred embodiment of the present invention provides a composition comprising a detectable agent for Prevotella spp., Sneathia spp. Megasphaera spp., Gardnerella spp., and Lactobacillus spp. and can detect the female genital disease or obesity with a high sensitivity by including all strains having the high detection specificity.

As another example of the present invention, the composition comprising a microorganism detecting agent of the present invention may be provided in the form of a kit for predicting or diagnosing the risk of female genital diseases or obesity. The kit of the present invention not only includes a detection agent for detecting the microorganisms, such as primers, probes, antisense oligonucleotides, aptamers and/or antibodies, as well as one or more other components, solutions, or devices which can be used suitable for the analyzing method.

The description for the composition of prediction or diagnosis of risk female genital diseases or obesity may be applied to a kit for prediction or diagnosis risk of female genital diseases or obesity.

As a specific example, a kit containing a primer specific to the microorganism in the present invention may be a kit containing essential elements for carrying out an amplification reaction such as PCR and the like. For example, the kit for PCR can be used in a test tube or other suitable container, reaction buffer, deoxynucleotides (dNTPs), enzymes such as Taq polymerase reverse transcriptase, DNase, RNAse inhibitor, DEPC-water, sterilized water, and the like.

The kit may be applied to a sample taken from the vagina of the subject. Specifically the sample taken in the vagina can be a vaginal discharge or amniotic fluid sample taken from the cervix.

The kit includes a collecting device for collecting the sample, for example, a collecting device for collecting a vaginal discharge or an amniotic fluid sample. The examples of collecting device include at least one collection device selected from the group consisting of a brush, an absorbent pad, a swab, a syringe, a spoon, and an amniotic fluid collector, but not limited thereto.

The kit may further include instructing description to require that the subject's sample be taken from the vagina for the purpose of predicting or diagnosing the risk of female reproductive disease or obesity.

As another example, in order to provide information required for predicting or diagnosing the risk of risk female genital diseases or obesity, the present invention provides a method of detecting at least a vaginal microorganism selected from the group consisting of Prevotella spp., Sneathia spp. Megasphaera spp., Gardnerella spp., and Lactobacillus spp.

The description relating to the composition for predicting or diagnosing the risk of female genital diseases or obesity may be similarly applied to the method for detecting microorganisms.

In a preferred embodiment, the method of the present invention comprises (a) step of collecting test sample in the vagina of the subject; (b) step of extracting genomic DNA from the sample; step of reacting the extracted genomic DNA with a specific primer to at least a vaginal microorganism selected from the group consisting of Prevotella spp., Sneathia spp. Megasphaera spp., Gardnerella spp., and Lactobacillus spp.; and (c) step of amplifying the reaction product.

In the above step (a), the term, “a test sample of a subject” refers to a sample taken from a subject who has is a female genital diseases or obesity, or expected to have a female genital diseases or obesity, for examples, tissue, cell, whole blood, serum, plasma saliva or urine, or preferably the vagina or cervix of subject.

The extraction of the genomic DNA from the test sample of the subject in the step (b) can be performed by applying a general technique known in the art. In step (c), a primer specific to at least one microorganism selected from the group consisting of a specific primer to at least a vaginal microorganism selected from the group consisting of Prevotella spp., Sneathia spp. Megasphaera spp., Gardnerella spp., and Lactobacillus spp.

The method of amplifying the reaction product in the step (c) may be carried out using conventional amplification techniques known in the art, for example, polymerase chain reaction (PCR), SYBR real time PCR, reverse transcription-polymerase chain reaction, multiplex PCR, touchdown PCR, PCR, nested PCR, booster PCR, real-time PCR, fractional display PCR, Rapid amplification of cDNA ends, Inverse PCR, VECTORET PCR, TAIL-PCR, Ligase chain reaction, Restriction chain reaction, Transcription-Selective replication of the target base sequence may be used, but the present invention is not limited thereto.

In the step (c), the step (e) of comparing the amount of amplification product of the test sample with the amount of amplification product of the normal control sample can be further performed, and the subject can be determined to have the presence of occurrence or high risk of female genital diseases or obesity.

Preferably, when the amplification product of at least a vaginal microorganism selected from the group consisting of Prevotella spp., Sneathia spp. Megasphaera spp., and Gardnerella spp. is higher than that of the normal control sample, the occurrence or the risk of female genital diseases or obesity may be predicted or diagnosed.

In addition, Prevotella spp., Sneathia spp. Megasphaera spp., and Gardnerella spp. are negatively correlated with Lactobacillus spp. which is good in the vaginal microbiota.

In a preferred embodiment, the present invention provides a method of screening a therapeutic agent or a prophylactic agent for a female genital disease or obesity, comprising treating with h the candidate substance for prevention or treatment of a female genital disease or obesity; detecting change in at least a vaginal microorganism selected from the group consisting of Prevotella spp., Sneathia spp. Megasphaera spp., Gardnerella spp., and Lactobacillus spp; and determining the candidate substance as a therapeutic agent or a prophylactic agent for a female genital disease or obesity, when the detecting amount of at least a vaginal microorganism selected from the group consisting of Prevotella spp., Sneathia spp., Megasphaera spp., and Gardnerella spp. is decreased, or when the detecting amount of Lactobacillus spp. is increased.

The description relating to the composition can be equally applied to the method of screening a therapeutic agent.

Specifically, the present invention may be applied to a method for comparing change in at least one microbial community selected from the group consisting of Prevotella spp., Sneathia spp. Megasphaera spp., Gardnerella spp. and Lactobacillus spp., in the test sample taken from the vagina of the subject, in the presence or absence of the candidate substance for prevention or treatment of female genital disease or obesity, and the method can be applied to the method of screening a therapeutic agent or a prophylactic agent for a female genital disease or obesity. In other words, the degree of forming the microbial cluster for each Prevotella spp., Sneathia spp. Megasphaera spp., Gardnerella spp. and Lactobacillus spp. in a sample is measured and compared, in the presence or absence of the candidate substance for prevention or treatment of female genital disease or obesity, and the candidate substance can be developed as a therapeutic agent or a prophylactic agent for a female genital disease or obesity, when the degree of forming the microbial cluster is decreased in the presence of candidate substance.

The candidate substance includes various compounds with low molecular weight, compounds with high molecular weight, nucleic acid molecules (for example, DNA, RNA, PNA, etc.), proteins, sugars and lipids which can be a therapeutic agent or a prophylactic agent for female genital disease or obesity, but is not limited thereto.

### Effect of Invention

The present invention can provide vaginal microorganisms of Prevotella spp., Sneathia spp. Megasphaera spp., Gardnerella spp., and Lactobacillus spp. as a new biomarker for the prediction or diagnosis of the risk of female genital diseases or obesity. The vaginal microorganisms can be utilized as a target of the development and control of new drugs for prevention or treatment of female genital health or obesity in the vagina with superior sensitivity compared to the conventional studies conducted with intestinal microorganisms. By using the combination of vaginal microorganisms having the high detection sensitivity, the female genital diseases or obesity can be more effectively predicted or diagnosed.

### BRIEF DESCRIPTION OF DRAWINGS

FIG. 1 shows the change in the distribution of vaginal microflora according to pre-menopausal/post-menopause, vaginitis (BV), cervical inflammation (Cervicits), cervical cancer (HPV) and obesity (BMI>30), on the basis of 16S rRNA-based analysis.

FIG. 2 shows NMDS plots of clinical indices that can significantly change the structure of microbiota in vagina.

FIG. 3 shows the result of evaluating the diversity of vaginal flora in obesity in pre-menopausal and post-menopausal vagina.

FIG. 4 shows the change in specific vaginal bacteria according to menopausal status.

FIG. 5 shows the change in specific vaginal bacteria according to vaginitis infection.

FIG. 6 shows the change in specific vaginal bacteria according to obesity.

FIG. 7 shows the change in specific vaginal bacteria according to the treatment of female hormone.

FIG. 8 shows the change in specific vaginal bacteria according to cervical cancer.

FIG. 9 shows the result confirming the occurrence pattern of vaginal microflora in the pre-menopausal vagina using SparCC software.

FIG. 10 shows the result confirming the occurrence pattern of vaginal microflora in post-menopausal vagina using SparCC software.

FIG. 11 shows the result of analyzing the diversity in the vaginal microflora of the rats fed the normal diet and the obese rats fed the high-fat diet.

FIG. 12 shows the result of univariate analysis of vaginal microflora according to the diet.

FIG. 13 shows the change in the blood lipopolysaccharide of the rats fed with a normal diet, which was administered by the vaginal microflora obtained from the rats fed with high-fat diet.

### MODE OF INVENTION

Hereinafter, the present invention will be described in detail with reference to examples. However, the following examples illustrate the present invention, and the present invention is not limited by the following examples.

### Example 1. Study Subject and Sample Collection

For the present invention, samples in the endocervix were collected with brush from 542 female identical tweens sand fraternal twins from Korean tween cohorts, and their families, and stored in a frozen state at −80° C. degrees. The cryopreserved samples were transferred to the laboratory and genomic DNA was extracted using a kit.

### Example 2. Analysis of Vaginal Microflora Using 16S rRNA

The genomic DNA extracted from Example 1 was amplified using the 515F/806R primers (SEQ ID NOs: 1 and 2) shown in Table 1, targeting the V4 region of the bacteria, and followed by nucleotide sequence data using Illumina's MiSeq instrument.

TABLE 2

| | SEQ | |
|---|---|---|
| | ID | |
| Item | NO | Nucleotide sequence |
| Forward | 1 | 5′-AATGATACGGCGACCACCGAGATCTACACTATGGT |
| primer | | AATTGTGTGCCAGCMGCCGCGGTAA-3′ |
| Reverse | 2 | 5′-CAAGCAGAAGACGGCATACGAGATAGTCAGTCAGC |
| primer | | CGGACTACHVGGGTWTCTAAT-3′ |

By using the QIIME pipeline, the analysis of nucleotide sequence data were performed to confirm the genetic information and the structure of vaginal microflora, and then the relationship between the vaginal microflora and the various female health markers were observed.

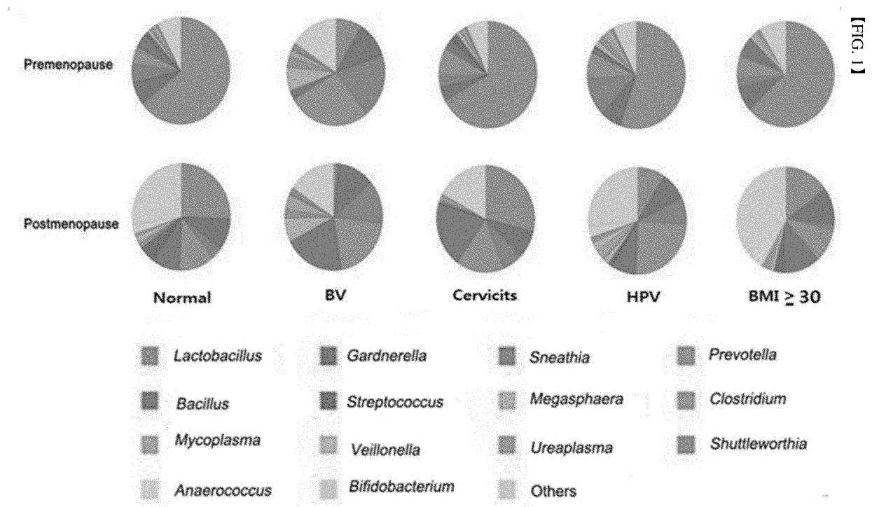

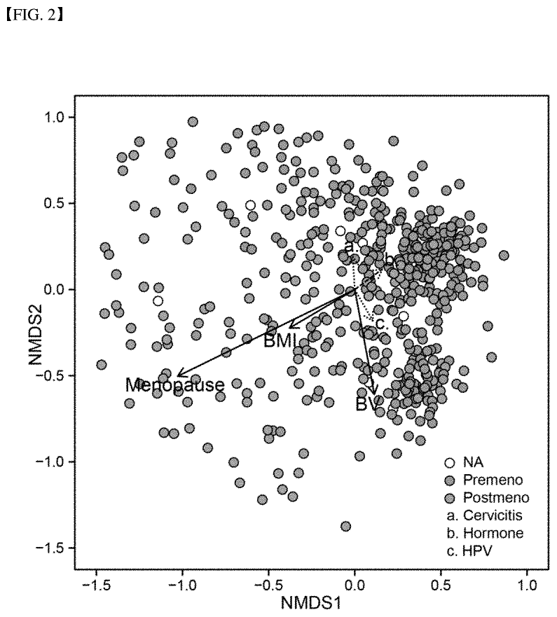

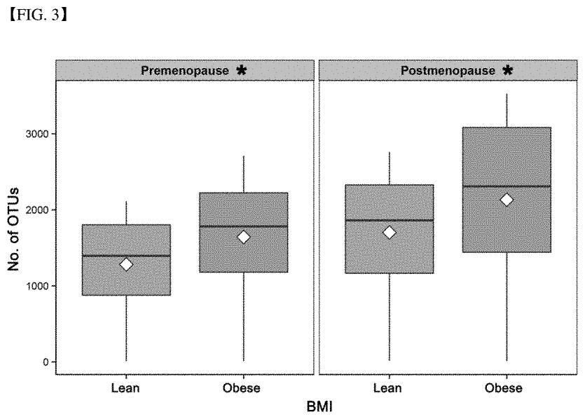

The vaginal microflora information obtained by 16S rRNA-based analysis confirms the changes in the vaginal microflora according to menopausal status, vaginitis, cervical inflammation, cervical cancer and obesity. The results are shown in FIG. 1. As a result of analyzing the association of factors, it was confirmed that clinical indicators that can significantly change the structure of the vesicles in vagina were menopause, vaginitis, and obesity (FIG. 2). As a result of analyzing the correlation of indicators affecting health with the vaginal microflora by using NMDS plots, the clinical indictors that can significantly change the structure of microbiota in vagina were post-menopausal status, vaginitis infection, and obesity (FIG. 2) In addition, it was confirmed that the obesity increased the diversity vaginal microflora (FIG. 3). That suggests that the harmful vaginal microorganisms induced by the obesity may have a negative influence on female genital health, by considering that the beneficial bacteria, Lactobacillus is dominant species in the vaginal microflora.

### Example 3. Analysis of Correlation Between Vaginal Microflora and Female Genital Diseases or Obesity

The result of multivariate analysis with MaAsLin software, which can control confounding variables, vaginal bacteria identifying female genital diseases or obesity was defined.

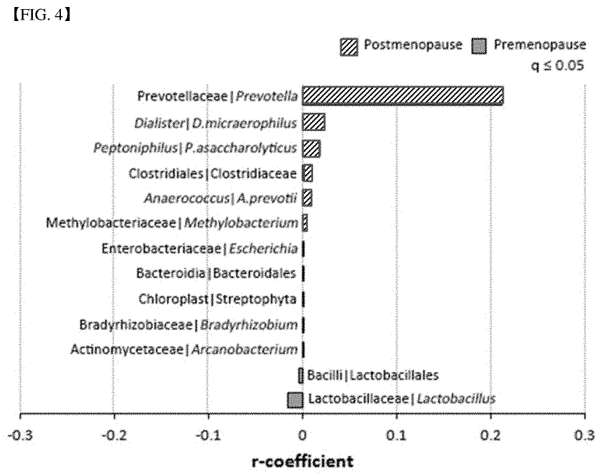

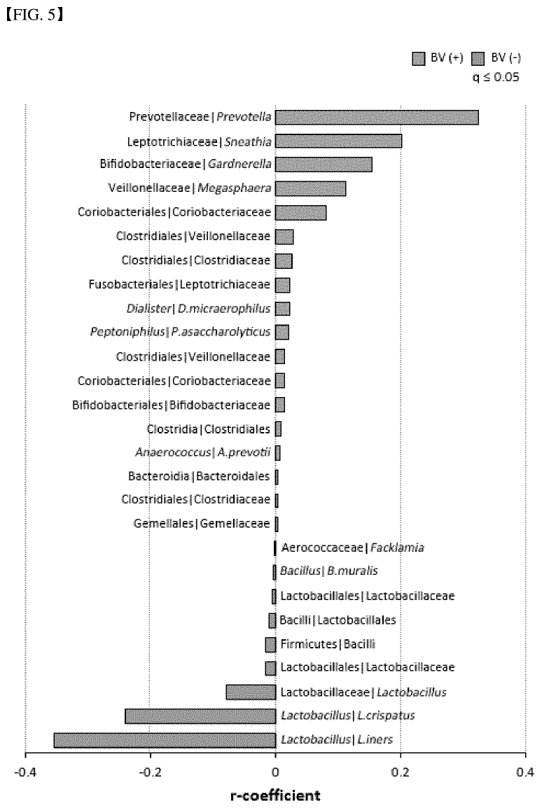

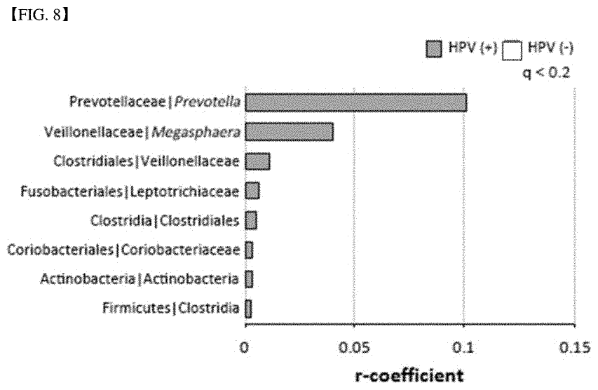

Using the MaAsLin software, the twin and family relationships were assigned as random variables and other health factors as calibration variables, and the correlation between each health factor and vaginal microflora was analyzed. The results are shown in FIGS. 4 to 8.

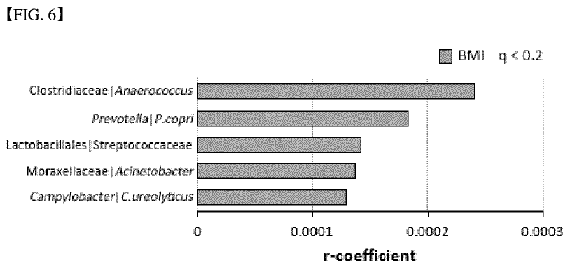

As can be seen from FIGS. 4, 5 and 8, it was found that Prevotella spp. was highest correlation with the female genital diseases, especially menopause, vaginitis and cervical cancer. In the case of obesity, P. copri, one of the species of Prevotella spp. was found to be the secondly-high correlation to the obesity (FIG. 6).

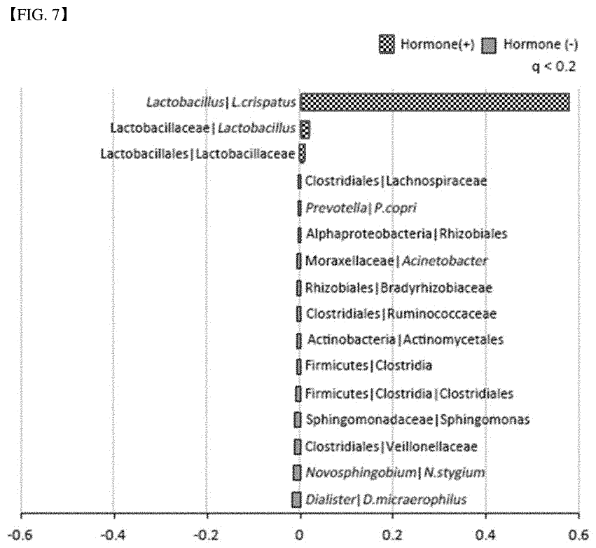

Besides the post-menopausal status, vaginitis infection, and obesity showing the significant correlation in NMDS plot, the female hormone therapy had a negative correlation (r correlation coefficient=−0.004, q-value: 0.18) (FIG. 7) and HPV infection had a positive correlation (r correlation coefficient=0.1, q-value: 0.16).

### Example 4. Network Analysis of Vaginal Microflora

The network analysis was performed using Sparse Correlations for Compositional Data (SparCC) software to determine the pattern of occurrence of vaginal microflora in female genital or obesity groups and normal groups.

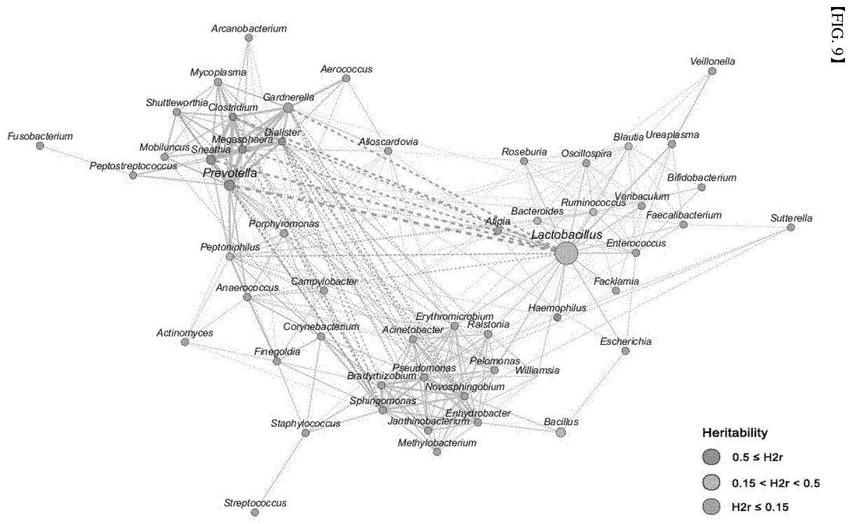

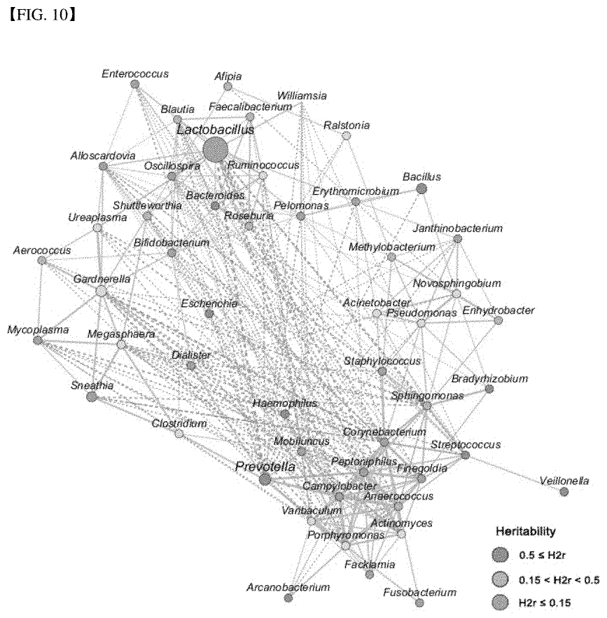

As a result, the inter-relationship between vaginal microflora was observed through additional network analysis. It was found that Prevotella spp. was highest correlation with Lactobacillus spp. in both pre-menopausal (FIG. 9) and post-menopausal (FIG. 10).

### Example 5. Animal Experiments to Confirm the Relationship Between Female Vaginal Microflora and Obesity

Animal experiments were conducted to investigate the relationship between obesity and female vaginal microflora. Six week old C57BL/6J rats were fed high fat diets for 12 weeks and the vaginal microflora were analyzed.

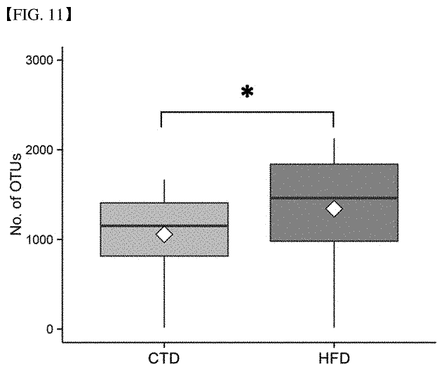

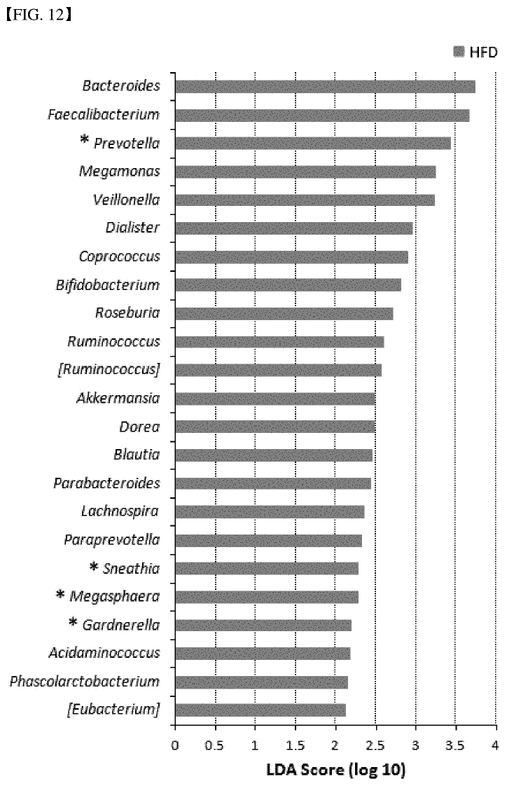

Analysis of vaginal microflora in obese rats fed with high-fat diets resulted in increased diversity of vaginal microflora (FIG. 11). Specifically, the univariate analysis of vaginal microflora depending on the diet showed that the Gram-negative bacteria, Prevotella spp., Sneathia spp. Megasphaera spp. and Gardnerella spp. which are known as the harmful group to the human vaginal environment have been significantly increased (FIG. 12).

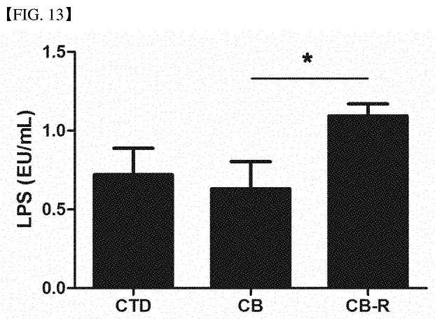

In addition, in order to investigate the effect of the obesity-altered vaginal microflora on the host, the vaginal microflora of obese rats fed with high fat diet were obtained and administered five times into the vagina of normal rats fed with general diet. The blood was obtained from the administered normal rats and was measured for the lipopolysaccharide constituting the cell wall of Gram-negative bacteria to show the result in FIG. 13. As a result, the lipopolysaccharide was significantly increased in the blood of the normal rats administered by the vaginal microflora of obese rats fed with high fat diet. This suggests that Gram-negative organisms induced by obesity may have deleterious effects on the whole body.

1. A composition for predicting or diagnosing presence or risk of female genital diseases or female obesity, comprising a detectable agent of at least a vaginal microorganism selected from the group consisting of Prevotella spp., Sneathia spp. Megasphaera spp., Gardnerella spp., and Lactobacillus spp.

2. The composition according to claim 1, wherein the female genital diseases are at least one selected from vaginitis, menopause, cervical inflammation and cervical cancer.

3. The composition according to claim 1, wherein the detectable agent of microorganism is a primer, a probe, an antisense oligonucleotide, an aptamer or an antibody which is specific to the microorganism.

4. A kit for predicting or diagnosing risk of female genital diseases or female obesity, using a vaginal microorganism, comprising a composition of claim 1.

5. The kit according to claim 4, wherein the kit is applied to a sample collected from the vagina of a subject.

6. The kit according to claim 5, wherein the sample collected from the vagina is vaginal secretion or amniotic fluid.

7. The kit according to claim 6, wherein the vaginal secretion is obtained from vagina or cervix.

8. The kit according to claim 6, wherein the kit further comprises a sample collection device for taking a sample from an vaginal secretion or an amniotic fluid of a subject, and the sample collection device is selected from the group consisting of a brush, an absorbent pad, a swab, a syringe, a spoon, and an amniotic fluid collector.

9. A method of prediction or diagnosis of presence or risk of female genital diseases or female obesity, comprising a step of detecting at least a vaginal microorganism selected from the group consisting of Prevotella spp., Sneathia spp. Megasphaera spp., Gardnerella spp., and Lactobacillus spp.

10. The method according to claim 9, wherein the method comprises the steps of:
(a) collecting test sample in the vagina of the subject;
(b) extracting genomic DNA from the sample
(c) reacting the extracted genomic DNA with a specific primer to at least a vaginal microorganism selected from the group consisting of Prevotella spp., Sneathia spp. Megasphaera spp., Gardnerella spp., and Lactobacillus spp. and
(d) amplifying the reaction product.

11. The method according to claim 10, wherein the sample collected from the vagina is vaginal secretion or amniotic fluid.

12. The method according to claim 10, further comprising step (e) of comparing the amplification product amount of the genomic DNA of the sample with the amplification product amount of the normal control sample, after step (d).

13. The method according to claim 12, wherein the presence of occurrence or risk of female genital diseases is determined, when the amplification product amount of at least a vaginal microorganism selected from the group consisting of Prevotella spp., Sneathia spp. Megasphaera spp. and Gardnerella spp. in a test sample is higher than that of normal control sample.

14. The method according to claim 12, wherein the presence of occurrence or risk of female genital diseases is determined, when the amplification product amount of Lactobacillus spp. in a test sample is lower than that of normal control sample.

15. A method of screening a prophylactic or therapeutic agent for female genital diseases or female obesity, comprising:
treating with a candidate substance for the therapeutic or prophylactic agent,
detecting change in at least a vaginal microorganism selected from the group consisting of Prevotella spp., Sneathia spp. Megasphaera spp., Gardnerella spp. and Lactobacillus spp. in samples obtained from vagina before and after treating with a candidate substance, and
determining the candidate substance as a therapeutic or prophylactic agent, when the detecting amount of at least a vaginal microorganism selected from the group consisting of Prevotella spp., Sneathia spp. Megasphaera spp., and Gardnerella spp. in a test sample decreases, or the detecting amount of Lactobacillus spp. increase, after treating with a candidate substance.

16-29. (canceled)

30. The kit according to claim 4, wherein the female genital diseases are at least one selected from vaginitis, menopause, cervical inflammation and cervical cancer.

31. The kit according to claim 4, wherein the detectable agent of microorganism is a primer, a probe, an antisense oligonucleotide, an aptamer or an antibody which is specific to the microorganism.

32. The method according to claim 11, wherein the sample collected from the vagina is vaginal secretion or amniotic fluid.
